(19) United States
(12) Patent Application Publication
(10) Pub. No.: US 2019/0043533 A1
(43) Pub. Date: Feb. 7, 2019

(54) SYSTEM AND METHOD FOR EFFECTUATING PRESENTATION OF CONTENT BASED ON COMPLEXITY OF CONTENT SEGMENTS THEREIN

(71) Applicant: KONINKLIJKE PHILIPS N.V., Eindhoven (AE)

(72) Inventors: Anand SRINIVASAN, Chennai (IN); Rithesh SREENIVASAN, Bangalore (IN); Rajendra Singh SISODIA, Bhopal (IN)

(21) Appl. No.: 16/061,392

(22) PCT Filed: Dec. 20, 2016
(86) PCT No.: PCT/EP2016/082015

§ 371 Date: Jun. 12, 2018

(30) Foreign Application Priority Data
Dec. 21, 2015 (IN) 6801/CHE/2015

(51) Int. Cl. G11B 27/031; G06F 17/30; G16H 50/20; G06K 9/00; G06F 17/27; G09B 5/06

(52) U.S. Cl. CPC G11B 27/031; G06F 17/30817; G16H 50/20; G09B 5/06; G06K 9/00718; G06F 17/2785; G06K 9/00765

(57) ABSTRACT

This disclosure describes a system that effectuates presentation of video content based on complexity of video content segments therein. The system may analyze the video content using semantic ontology to identify semantic concepts; segment the video content into one or more video content segments based on identified semantic concepts, determine the complexity measure of the one or more video content segments based on a weightage of the identified semantic concepts, present the one or more video content segments based on the complexity measure, and present a visualization of the measure of the complexity.

### BACKGROUND

### 1. Field

The present disclosure pertains to a system and method for effectuating presentation of content, for example, based on complexity of content segments therein.

### 2. Description of the Related Art

Coaching a user during presentation of content to the user is an effective means in helping the user to understand a topic that needs to be understood by the user. Such coaching can relate to different and varying topics such as health care and education, and be used to facilitate e-learning. The content for coaching may be in the form of a video, text, audio and/or other forms.

### SUMMARY

Accordingly, one or more aspects of the present disclosure relate to a system configured to effectuate presentation of video content based on complexity of video content segments therein. The system comprises one or more hardware processors and/or other components. The one or more hardware processors are configured by machine-readable instructions to analyze the video content using semantic ontology to identify semantic concepts in the video content, an individual semantic concept indicated by a plurality of linked keywords corresponding to an individual topic of the video content; segment the video content into one or more video content segments based on the semantic concepts; determine a measure of complexity of the video content based on a weightage of the identified semantic concepts, the weightage of the identified semantic concepts determined based on one or more of types or numbers of links associated with the identified semantic concepts, numbers of concept nodes associated with the identified semantic concepts, the number of concept nodes indicating a number of other semantic concepts relating a given semantic concept, an education level corresponding to the user, evaluation results of the user responding to a query or survey relating to the content, or a clinical medical condition of the user; and; effectuate presentation of the one or more video content segments to the user based on the determination of the measure of complexity of the video content.

Another aspect of the present disclosure relates to a method for effectuating presentation of video content based on complexity of video content segments therein. The system comprises one or more hardware processors and/or other components. The method comprises analyzing the video content using semantic ontology to identify semantic concepts in the video content, an individual semantic concept indicated by a plurality of linked keywords corresponding to an individual topic of the video content; segmenting the video content into one or more video content segments based on the semantic concepts; determining a measure of complexity of the video content based on a weightage of the identified semantic concepts, the weightage of the identified semantic concepts determined based on one or more of types or numbers of links associated with the identified semantic concepts, numbers of concept nodes associated with the identified semantic concepts, the number of concept nodes indicating a number of other semantic concepts relating a given semantic concept, an education level corresponding to the user, evaluation results of the user responding to a query or survey relating to the content, or a clinical medical condition of the user; and effectuating presentation of the one or more video content segments to the user based on the determination of the measure of complexity of the video content.

Still another aspect of present disclosure relates to a system for effectuating presentation of video content based on complexity of video content segments therein. The system comprises: means for analyzing the video content to identify semantic concepts in the video content, an individual semantic concept indicated by a plurality of linked keywords corresponding to an individual topic of the video content; means for segmenting the video content into one or more video content segments based on the semantic concepts; means for determining a measure of complexity of the video content based on a weightage of the identified semantic concepts, the weightage of the identified semantic concepts determined based on one or more of types or numbers of links associated with the identified semantic concepts, numbers of concept nodes associated with the identified semantic concepts, the number of concept nodes indicating a number of other semantic concepts relating a given semantic concept, an education level corresponding to the user, evaluation results of the user responding to a query or survey relating to the content, or a clinical medical condition of the user; and means for effectuating presentation of the one or more video content segments to the user based on the determination of the measure of complexity of the video content.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 is a schematic illustration of a system configured to effectuate presentation of content.

FIG. 2 illustrates a concept tree pertaining to heart failure according to one or more embodiments.

FIG. 3 illustrates a heart failure video being processed according to one or more embodiments.

FIG. 4 illustrates visualization of complexity in a timeline view according to one or more embodiments.

FIG. 5 illustrates a method for effectuating presentation of content based on complexity of video content segments therein.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

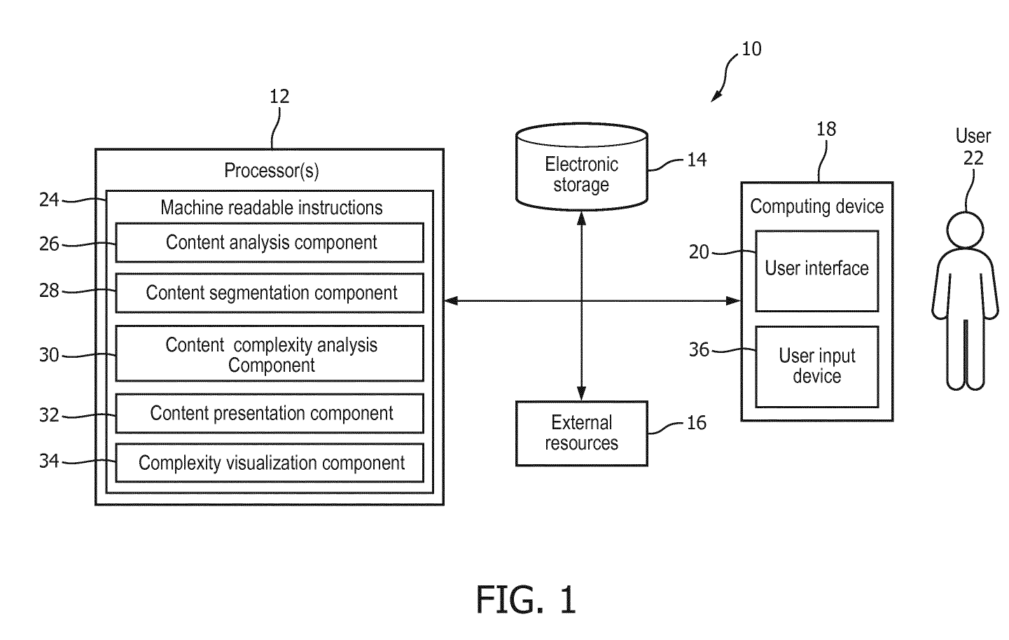

FIG. 1 is a schematic illustration of a system 10 configured to effectuate presentation of content. System 10 facilitates a better understanding of the concepts and topics in the delivered content. System 10 is configured to provide coaching to the user with varying complexity measures corresponding to the delivered the content, such that the delivered content may become more effective because of the content being customized to an understanding capability of user 22.

Present methods used to deliver content to users do not involve correlating a measure of complexity of the content with various interactions a user may have before, during, and after the presentation of the content. Present approaches are not specific to a particular user. Moreover, these approaches do not facilitate determining a depth and/or breadth of understanding of concepts discussed in the content by the user.

Present content delivery techniques were not designed with flexibility for content adjustment during the course of coaching through content delivery. For example, a user might press play on a playback device and then listen to and/or watch predetermined presented content. Thus the extent to which the content can be altered and/or rearranged and/or modified with such techniques is limited (e.g., if the user replayed the content, the user would see and/or hear the exact same presentation again). This approach is not tailored to a specific user, and results in a lack of effectiveness in meeting the goal for which the content is presented and/or the user is coached.

System 10 ensures that a user has understood the content and/or the information conveyed through the content at specific instances, before proceeding further so as to make the coaching meaningful and effective in meeting the goal and/or the purpose for which the user is exposed to such content.

System 10 is configured to analyze and segment the content based on semantic concepts present in the content, measure the complexity of the content based on at least one complexity parameter, and provide the content to user 22 based on the complexity of the content. In some embodiments, system 10 analyzes content using semantic ontology to identify semantic concepts; segments the content into one or more content segments based on identified semantic concepts; determines the complexity measure of the content based on a weightage of the identified semantic concepts; and presents the one or more content segments based on complexity measure. For example, content corresponding to heart failure may be presented to the user. In this example, system 10 may analyze the content to identify semantic concepts (topics) such as heart attack, high blood pressure, and/or other semantic concepts. System 10 may segment the content into one or more content segments based on the previously identified semantic concepts (e.g. heart attack, high blood pressure) and associate segments including similar topics into a sequence of topics for presentation. In this example, system 10 may determine the complexity measure of previously identified topics by adding the number of concept nodes (described below) with a weightage assigned to each semantic concept increased by one. System 10, presents the one or more content segments based on the determined complexity measure of the semantic concepts such that more difficult and/or challenging semantic concepts are presented when the user has understood less difficult and/or basic concepts. In some embodiments, system 10 comprises one or more of a processor 12, electronic storage 14, external resources 16, a computing device 18, and/or other components.

Processor 12 is configured to provide information processing capabilities in system 10. As such, processor 12 may comprise one or more of a digital processor, an analog processor, a digital circuit designed to process information, an analog circuit designed to process information, a state machine, and/or other mechanisms for electronically processing information. Although processor 12 is shown in FIG. 1 as a single entity, this is for illustrative purposes only. In some embodiments, processor 16 may comprise a plurality of processing units. These processing units may be physically located within the same device (e.g., a server), or processor 12 may represent processing functionality of a plurality of devices operating in coordination (e.g., a server, computing device 18 associated with user 22, devices that are part of external resources 16, and/or other devices.)

As shown in FIG. 1, processor 12 is configured via machine-readable instructions 24 to execute one or more computer program components. The one or more computer program components may comprise one or more of a content analysis component 26, a content segmentation component 28, a content complexity analysis component 30, a content presentation component 32, a complexity visualization component 34, and/or other components. Processor 12 may be configured to execute components 26, 28, 30, 32, and/or 34 by software; hardware; firmware; some combination of software, hardware, and/or firmware; and/or other mechanisms for configuring processing capabilities on processor 12.

It should be appreciated that although components 26, 28, 30, 32, and 34 are illustrated in FIG. 1 as being co-located within a single processing unit, in embodiments in which processor 12 comprises multiple processing units, one or more of components 26, 28, 30, 32, and/or 34 may be located remotely from the other components. The description of the functionality provided by the different components 26, 28, 30, 32, and/or 34 described below is for illustrative purposes, and is not intended to be limiting, as any of components 26, 28, 30, 32, and/or 34 may provide more or less functionality than is described. For example, one or more of components 26, 28, 30, 32, and/or 34 may be eliminated, and some or all of its functionality may be provided by other components 26, 28, 30, 32, and/or 34. As another example, processor 12 may be configured to execute one or more additional components that may perform some or all of the functionality attributed below to one of components 26, 28, and/or 30.

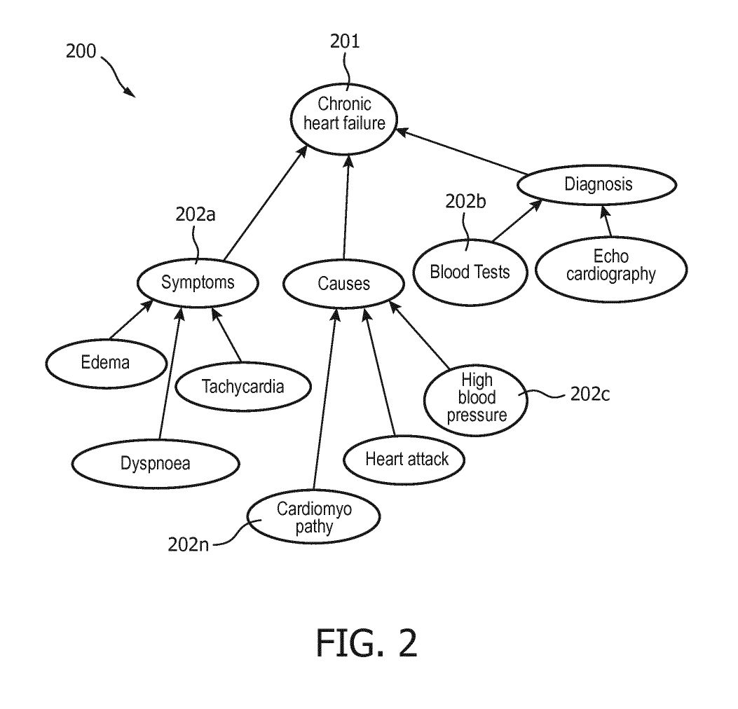

Content analysis component 26 is configured to analyze the content using semantic ontology to identify semantic concepts in the content. The content may be a video and/or textual information (e.g., closed captions of a multimedia video), or information in any other form intended to provide information on a topic related to a clinical condition and/or a clinical plan or goal. Semantic concepts include and/or refer to different topics included in the content. In some embodiments, an individual semantic concept may be indicated by a plurality of linked keywords corresponding to an individual topic of the content. In some embodiments, semantic ontology is a lexical database which groups words into sets of synonyms, records relations among the synonyms, and provides short definitions and usage examples regarding the synonyms. In some embodiments, semantic ontology may include nomenclature of medicine and clinical terms. In some embodiments, semantic ontology may include a dictionary and thesaurus. For example, content analysis component 26 may utilize a semantic ontology (e.g. WordNet™ for non-medical concepts or SNOMED-CT™ for medical concepts) to identify semantic concepts (e.g. Heart Failure) in the closed caption of the multimedia video. Individual semantic concepts may have a varying degree of complexity, and may be related to other semantic concepts within the same content. In some embodiments, the interrelated semantic concepts may be visualized and/or analyzed in a tree form having various topics and keywords representing individual semantic concepts. By way of a non-limiting example, FIG. 2 illustrates a concept tree 200 pertaining to heart failure (e.g., an individual semantic concept) according to one or more embodiments. The semantic concept tree 200 has various topics 202a, 202b, 202c, . . . , 202n with different hierarchical levels with respect to one another and varying degrees of complexity. FIG. 2 illustrates, a semantic concept tree 200 pertaining to chronic heart failure generated by content analysis component 26 utilizing medical ontologies to analyze content presented to a user. In this example, the semantic concept of chronic heart failure 201 comprises various semantic concepts that are structurally and logically arranged and linked based on relevancy, dependency and applicability. Each of the semantic concepts 202a, 202b, 202c, . . . , 202n belongs to the semantic concept 201 (e.g., chronic heart failure). However, one or more of semantic concepts 201, 202a, 202b, . . . , 202n may be relevant and have links to other semantic concepts. The semantic concepts may be defined as nodes that have links coming into as well as going out of each of the semantic concepts. An increased number of links branching from other semantic concepts increases the complexity of a given semantic concept. In some embodiments, a hierarchy of a given semantic concepts at which the given semantic concept appears may affect the complexity of the given semantic concept. In some embodiments, each semantic concept may have its own concept tree. For example, the semantic concepts of heart attack, high blood pressure, and cardiomyopathy appearing in concept tree 200 at a lower hierarchical level may each have an individually distinct concept tree. As illustrated in FIG. 2, semantic concepts Edema, Tachycardia, and Dyspnoea are related to semantic concept Symptoms; semantic concepts Cardiomyopathy, Hear Attack, and High Blood Pressure are related to semantic concept Causes; and semantic concepts Blood Tests and Echo Cardiography are related to semantic concept Diagnosis. In this example, semantic concepts Symptoms, Causes, and Diagnosis are at a higher hierarchical level with respect to their corresponding related semantic concepts and represent more difficult topics.

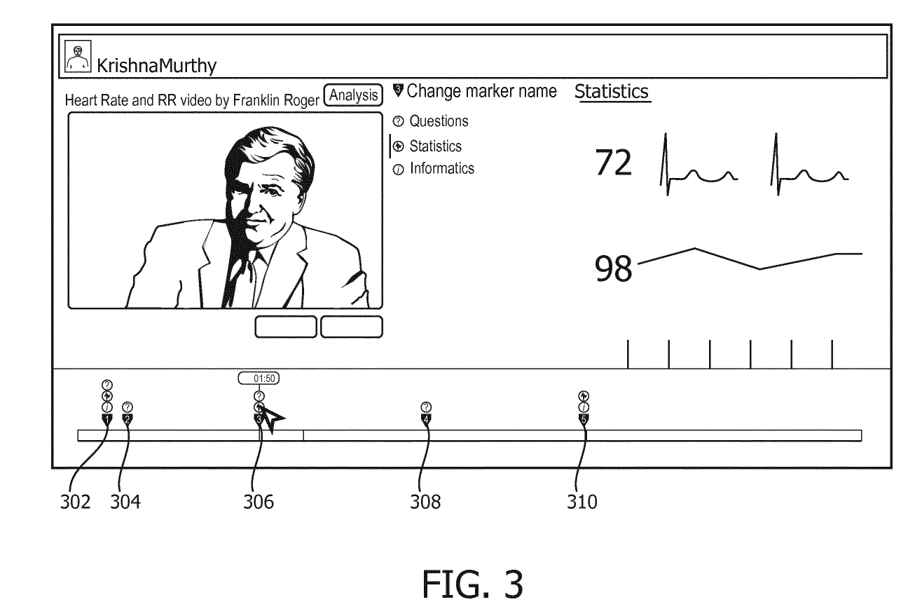

Returning to FIG. 1, content segmentation component 28 is configured to segment the content into one or more content segments based on the semantic concepts. In some embodiments, segmenting of content may be based on a semantic approach wherein content is segmented based on keywords that have a relevancy to the content and/or other relative content. Segmenting content may include dividing content into separate sections based on individual identified semantic concepts. In some embodiments, content segmentation component 28 identifies suitable time markers for presenting questions regarding individual content segments. In some embodiments, suitable markers may be identified for an expert user (e.g. clinician or physician) to provide relevant questions corresponding to the identified semantic concepts. This may be used to further evaluate and present the instruction based on the complexity and user 22's grasp of the content. By way of a non-limiting example, FIG. 3 illustrates a heart failure video being processed and various segments of the video being analyzed and determined using semantic analysis based algorithms on the text content of the video. In this example markers 302, 304, 306, 308, and 310 are identified for the clinicians to provide relevant questions related to the identified semantic concepts (e.g. Heart Rate, Effects of Smoking on Blood Pressure, Post Effect of Pacemaker).

Returning to FIG. 1, in some embodiments, content segmentation component 28 is configured to associate at least two semantic concepts based on an association parameter. Associating content may include merging content segments that are similar with respect to topic and/or content segments including similar keywords into a continuous sequence of content segments. In some embodiments, the association parameter may include one or more of individual topics of the content, links between the at least two semantic concepts (e.g., edges connecting nodes of a ontology graph where the nodes respectively represent the semantic concepts), and/or other parameters. The associating link may indicate the pre-condition to understand before the other concept can be understood. In some embodiments, the associating link may also indicate a path to traversal from simpler to complex concepts.

The content complexity analysis component 30 is configured to determine a measure of complexity of the one or more content segments based on a weightage of the identified semantic concepts. In some embodiments, the weightage of the identified semantic concepts may be determined by the hierarchical level of the identified semantic concepts. For example, as illustrated in FIG. 2, Chronic Heart Failure is identified as a disease semantic concept and has a higher hierarchical level with respect to Symptoms and Diagnosis semantic concepts; thus, Disease is assigned a higher weightage than Symptoms. In some embodiments, the weightage of the identified semantic concepts may be determined based on one or more of types or numbers of links associated with the identified semantic concepts, numbers of concept nodes associated with the identified semantic concepts, the number of concept nodes indicating a number of other semantic concepts relating a given semantic concept, an education level corresponding to the user, evaluation results of the user responding to a query or survey relating to the content, a clinical medical condition of the user, and/or other information. By way of a non-limiting example, Table 1 illustrates possible weightages corresponding to semantic concepts.

TABLE 1

| Semantic Type | Weightage |
|---|---|
| Symptom | 1 |
| Diagnostic Test | 1 |
| Physiologic Function | 2 |
| Disease | 2 |

In some embodiments, the measure of complexity is determined by adding the number of the concept nodes to a sum of a complexity measure of each of the identified semantic concepts. In some embodiments, the complexity measure of each of the identified semantic concepts may be determined by increasing a weightage of each of the identified semantic concepts by one. In some embodiments, measure of complexity may be determined using hop length method wherein the terms of a semantic concept are utilized to determine basic, intermediate and advanced semantic concepts in the content. For example, addition (e.g. 2+3) may be a simple concept in mathematics, and multiplication may be a concept that depends on addition (e.g. 5×5=(2+3)×(2+3)); thus multiplication is more complex than addition. In this example, until user 22 has understood the concept of addition, user 22 may find the concept of multiplication difficult to comprehend. In some embodiments, measure of complexity may be hop length indicated by a depth and a breadth of a concept. In some embodiments, a depth of a semantic concept is determined by a number of parent semantic concepts. In some embodiments, a breadth of a semantic concept is determined by a number of links associated with a semantic concept. For example, user 22 has to understand blood circulation, oxygenation flow, and muscular function of heart semantic concepts before learning heart failure semantic concept. In some embodiments, determination of the complexity measure of each of the identified semantic concepts, may include a two-step process. The two step process may include a) determining the complexity measure of each of the identified semantic concepts based on a domain knowledge point of view; and b) determining the complexity measure of each of the identified semantic concepts based on a user's point of view. Determination of the complexity measure based on a domain point of view includes measuring the complexity of each of the identified semantic concepts based on a corresponding weightage as determined by the semantic ontology relative to one or more of a discipline, a field of study, and/or a subject area. Determination of the complexity measure based on a user's point of view includes measuring the complexity based of each of the identified semantic concepts based on one or more of the user's education level, the user's prior exposure to each of the identified semantic concepts, the user's scientific knowledge about each of the identified semantic concepts and/or other factors. For example, to a user who has never been exposed to a given field, a simpler concept, as measured based on domain knowledge, may be complex, and to an expert user, a complex concept, as measured based on domain knowledge, may be a simpler concept.

By way of a non-limiting example, Table 2 illustrates complexity measured for various semantic concepts in the video (e.g., content) wherein a combination of number of measurable parameters is used to determine the overall complexity measure of each of these concepts. User 22 may be a patient with chronic heart failure and a lower level of education. In this example, it may be required for user 22 to understand the individual semantic concepts relating to heart rate, ankle swelling, and/or other semantic concepts from the video in order to understand heart failure. In some embodiments, overall complexity measure of individual semantic concepts may be illustrated by numbers (e.g. 1 for simple semantic concepts, 2 for difficult semantic concepts, 3 for very challenging semantic concepts). In some embodiments, overall complexity measure of individual semantic concepts may be illustrated by skill level (e.g. Basic, Intermediate, Advanced). In some embodiments, the content complexity analysis component 30 may illustrate video semantic graph transition probabilities.

TABLE 2

| | Depth of the | Breath of the | | | | |
|---|---|---|---|---|---|---|
| Semantic | semantic concept | semantic concept | Related semantic | Hop Length | Video Semantic | Overall |
| Concept in | in the ontology | in the ontology | concepts | for related | Graph Transition | Complexity |
| the Video | tree | tree | in the video | semantic concept | Probabilities | Measure |
| Heart rate | 4 | 5 | — | — | HIGH | ADVANCED |
| | concepts | concepts | | | | |
| Ankle | 1 | 2 | Heart Rate, | 2, 3 | MEDIUM | ADVANCED |
| Swelling | concept | concepts | Blood Pressure | | | |
| Blood | 2 | 4 | — | — | HIGH | INTERMEDIATE |
| pressure | concepts | concepts | | | | |
| Pacemakers | 1 | 2 | — | — | LOW | BASIC |
| | concept | concepts | | | | |

Returning to FIG. 1, content presentation component 32 is configured to present the one or more content segments to user 22 and/or other users. The one or more content segments may be displayed on computing device 18 and/or other devices. Computing device 18 comprises a user interface 20 and/or other components. Computing device 18 facilitates presentation of the content to user 22. Computing device 18 comprises a user input device 36 facilitating entering and/or selecting responses by user 22. In some embodiments, user input device 36 includes a mouse, a touchscreen, and/or other components (e.g., as described below related to computing device 18) facilitating selecting an answer choice in a multiple choice query or survey and a keyboard (and/or other components as described below) facilitating typing answers to a corresponding query or survey.

Content presentation component 32 is configured to effectuate presentation of the one or more content segments to user 22 based on the determination of the measure of complexity of the one or more content segments. In some embodiments, effectuating presentation of the one or more content segments includes one or more of presenting rearranged, altered, modified, fragmented, combined, or replaced content segments. Rearranging content segments may include changing a presentation time of each of the content segments. Altering and/or modifying the one or more content segments may include changing textual or visual information corresponding to the content segments prior to presentation of the respective content segments. Fragmenting the one or more content segments may include dividing the content segments into smaller portions and presenting each of the smaller portions independent of one another. Combining content segments may include combining a plurality of similar content segments and/or other content segments prior to presentation of the content segments. Replacing content segments may include substituting one content segments for another content segment. For example, a content segment describing High Blood Pressure may be presented in fragmented components of nutrition, exercise, genetic heredity, and/or other components. In some embodiments, the analysis of the content to identify sematic concepts, the segmentation of the content, and/or the determination of the measure of complexity of the content or content segments therein may be performed prior to the presentation of the content or content segments therein. In some embodiments, the analysis of the content to identify sematic concepts, the segmentation of the content, and/or the determination of the measure of complexity of the content or content segments therein may be performed during at least a portion of the presentation of the content or content segments therein. As an example, the content or content segments to be presented to a user may be rearranged, altered, modified, fragmented, combined (e.g., with one or more other content segments), or replaced during presentation of at least a portion of the content to a user such that one or more rearranged, altered, modified, fragmented, combined, or replaced content or content segments may be presented to the user during the same presentation of the content in a dynamic fashion. In some embodiments,

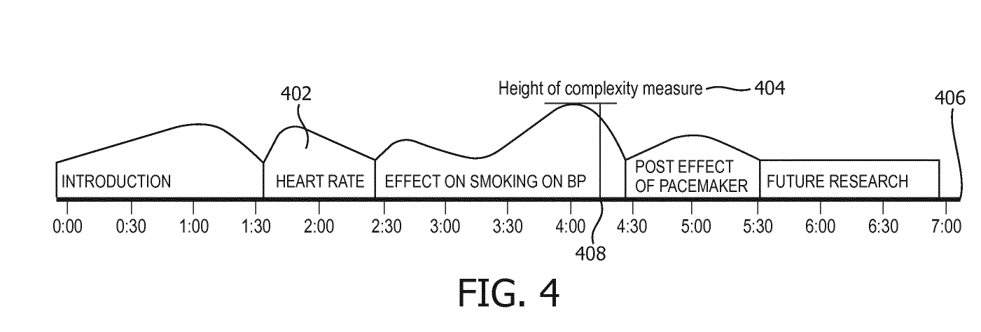

Complexity visualization component 34 is configured to, responsive to the determination of the measure of complexity of the identified semantic concepts and a timestamp corresponding to the identified semantic concepts, effectuate presentation of a visualization of the measure of complexity, the visualization including a statistical probability graph, a bar chart, or a timeline chart. By way of a non-limiting example, FIG. 4 illustrates visualization of complexity in a timeline view according to one or more embodiments. FIG. 4 illustrates the complexity of semantic concepts (e.g. semantic concept 402 pertaining to Heart Rate) plotted on timeline 406. In this example height of complexity 404 illustrates the complexity of the corresponding semantic concept at a particular time stamp 408.

Returning to FIG. 1, electronic storage 14 comprises electronic storage media that electronically stores information. The electronic storage media of electronic storage 14 may comprise one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 14 may be (in whole or in part) a separate component within system 10, or electronic storage 14 may be provided (in whole or in part) integrally with one or more other components of system 10 (e.g., computing device 18, processor 16, etc.). In some embodiments, electronic storage 14 may be located in a server together with processor 12, in a server that is part of external resources 16, in computing device 18 associated with user 22, and/or other users, and/or in other locations. Electronic storage 14 may comprise one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media. Electronic storage 14 may store software algorithms, information determined by processor 12, information received via computing device 18 and/or other external computing systems, information received from external resources 16, and/or other information that enables system 10 to function as described herein. By way of a non-limiting example, electronic storage 14 may store a user profile for user 22 and/or other information.

External resources 16 include sources of information (e.g., databases, websites, etc.), external entities participating with system 10 (e.g., a medical records system of a health care provider that stores a health plan for user 22), one or more servers outside of system 10, a network (e.g., the internet), electronic storage, equipment related to Wi-Fi technology, equipment related to Bluetooth® technology, data entry devices, computing devices associated with individual users, and/or other resources. For example, in some embodiments, external resources 16 may include the database where the medical records including medical conditions, symptoms, and/or other information relating to user 22 are stored, and/or other sources of information. In some implementations, some or all of the functionality attributed herein to external resources 16 may be provided by resources included in system 10. External resources 16 may be configured to communicate with processor 12, computing device 18, electronic storage 14, and/or other components of system 10 via wired and/or wireless connections, via a network (e.g., a local area network and/or the internet), via cellular technology, via Wi-Fi technology, and/or via other resources.

Computing device 18 is configured to provide an interface between user 22, and/or other users and system 10. Computing device 18 is configured to provide information to and/or receive information from the user 22, and/or other users. For example, computing device 18 is configured to present a user interface 20 to user 22 to facilitate presentation of multimedia video to user 22. In some embodiments, user interface 20 includes a plurality of separate interfaces associated with computing device 18, processor(s) 12 and/or other components of system 10.

In some embodiments, computing device 18 is configured to provide user interface 20, processing capabilities, databases, and/or electronic storage to system 10. As such, computing device 18 may include processor(s) 12, electronic storage 14, external resources 16, and/or other components of system 10. In some embodiments, computing device 18 is connected to a network (e.g., the internet). In some embodiments, computing device 18 does not include processor(s) 12, electronic storage 14, external resources 16, and/or other components of system 10, but instead communicate with these components via the network. The connection to the network may be wireless or wired. For example, processor(s) 12 may be located in a remote server and may wirelessly cause display of user interface 20 to user 22 on computing device 18. In some embodiments, computing device 18 is a laptop, a personal computer, a smartphone, a tablet computer, and/or other computing devices. Examples of user input device 36 suitable for inclusion in computing device 18 include a touch screen, a keypad, touch sensitive and/or physical buttons, switches, a keyboard, knobs, levers, a display, speakers, a microphone, an indicator light, an audible alarm, a printer, and/or other interface devices. The present disclosure also contemplates that computing device 18 includes a removable storage interface. In this example, information may be loaded into computing device 18 from removable storage (e.g., a smart card, a flash drive, a removable disk) that enables the user 22, and/or other users to customize the implementation of computing device 18. Other exemplary input devices and techniques adapted for use with computing device 18 include, but are not limited to, an RS-232 port, RF link, an IR link, a modem (telephone, cable, etc.) and/or other devices.

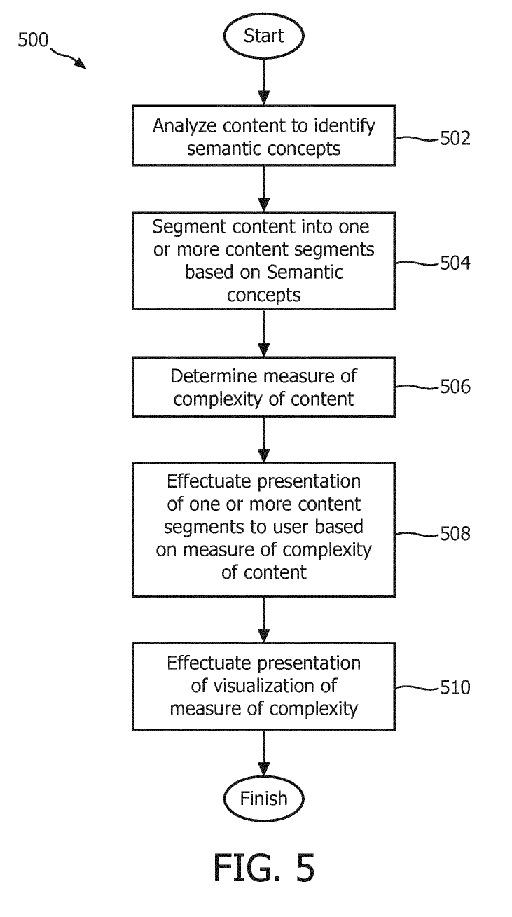

FIG. 5 illustrates a method 500 for determining complexity of content provided to a user having an interaction with the content with a system. The system comprises one or more hardware processors and/or other components. The one or more hardware processors are configured by machine readable instructions to execute computer program components. The computer program components comprise a content analysis component, a content segmentation component, a content association component, a content complexity analysis component, a content presentation component, and/or other components. The operations of method 500 presented below are intended to be illustrative. In some embodiments, method 500 may be accomplished with one or more additional operations not described, and/or without one or more of the operations discussed. Additionally, the order in which the operations of method 500 are illustrated in FIG. 5 and described below is not intended to be limiting.

In some embodiments, method 500 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 500 in response to instructions stored electronically on an electronic storage medium. The one or more processing devices may include one or more devices configured through hardware, firmware, and/or software to be specifically designed for execution of one or more of the operations of method 500.

At an operation 502, content is analyzed using semantic ontology to identify semantic concepts in the content. In some embodiments, an individual semantic concept may be indicated by a plurality of linked keywords corresponding to an individual topic of the content. In some embodiments, semantically analyzing the content includes automatically generating the query or survey and a recommended timestamp for effectuating presentation of the automatically generated query or survey. In some embodiments, operation 502 is performed by a processor component the same as or similar to content analysis component 26 (shown in FIG. 1 and described herein).

At an operation 504, content is segmented into one or more content segments based on the semantic concepts. In some embodiments, segmenting of the content based includes segmenting content using keywords relevant to the content. In some embodiments, segmenting content includes associating at least two semantic concepts with one another based on an association parameter. In some embodiments, the association parameter may include one or more of individual topics of the content, links between the at least two semantic concepts, and/or other parameters. In some embodiments, operation 504 is performed by a processor component the same as or similar to content segmentation component 28 (shown in FIG. 1 and described herein).

At an operation 506, a measure of complexity of the one or more content segments is determined based on a weightage of the identified semantic concepts. In some embodiments, the weightage of the identified semantic concepts may be determined based on one or more of types or numbers of links associated with the identified semantic concepts, numbers of concept nodes associated with the identified semantic concepts, the number of concept nodes indicating a number of other semantic concepts relating a given semantic concept, an education level corresponding to the user, evaluation results of the user responding to a query or survey relating to the content, a clinical medical condition of the user, and/or other information. In some embodiments, determining the measure of complexity of the content comprises adding the number of the concept nodes to a sum of a complexity measure of each of the identified semantic concepts. In some embodiments, the complexity measure of each of the identified semantic concepts may be determined by increasing a weightage of each of the identified semantic concepts by one. In some embodiments, operation 508 is performed by a processor component the same as or similar to content complexity component 30 (shown in FIG. 1 and described herein).

At an operation 508, the one or more content segments are presented to user based on the determination of the measure of complexity of the one or more content segments. In some embodiments, presenting the one or more content segments includes one or more of rearranging, altering, modifying, fragmenting, combining, and/or replacing the one or more content segments. In some embodiments, operation 508 is performed by a processor component the same as or similar to content presentation component 32 (shown in FIG. 1 and described herein).

At an operation 510, responsive to the determination of the measure of complexity of the identified semantic concepts and a timestamp corresponding to the identified semantic concepts, a visualization of the measure of complexity is presented. In some embodiments, the visualization includes a statistical probability graph, a bar chart, and/or a timeline chart. In some embodiments, operation 510 is performed by a processor component the same as or similar to complexity visualization component 34 (shown in FIG. 1 and described herein).

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

1. A system configured to effectuate presentation of video content based on complexity of video content segments therein, the system comprising one or more hardware processors configured by machine-readable instructions to:
analyze the video content using semantic ontology to identify semantic concepts in the video content, an individual semantic concept indicated by a plurality of linked keywords corresponding to an individual topic of the content;
segment the video content into one or more video content segments based on the semantic concepts;
determine a measure of complexity of the one or more video content segments based on a weightage of the identified semantic concepts, the weightage of the identified semantic concepts determined based on one or more of:
types or numbers of links associated with the identified semantic concepts,
numbers of concept nodes associated with the identified semantic concepts, the number of concept nodes indicating a number of other semantic concepts relating a given semantic concept,
an education level corresponding to the user,
evaluation results of the user responding to a query or survey relating to the content, or
a clinical medical condition of the user; and
effectuate presentation of the one or more video content segments to the user based on the measure of complexity.

2. The system of claim 1, wherein the one or more hardware processors are configured such that the measure of complexity is determined by adding the number of the concept nodes to a sum of a complexity measure of each of the identified semantic concepts, the complexity measure of each of the identified semantic concepts determined by increasing a weightage of each of the identified semantic concepts by one.

3. The system of claim 1, wherein the one or more hardware processors are configured such that effectuating presentation of the one or more video content segments includes one or more of rearranging, altering, modifying, fragmenting, combining, or replacing the one or more video content segments.

4. The system of claim 1, wherein the one or more hardware processors are further configured by machine-readable instructions to, responsive to the determination of the measure of complexity of the identified semantic concepts and a timestamp corresponding to the identified semantic concepts, effectuate presentation of a visualization of the measure of complexity, the visualization including a statistical probability graph, a bar chart, or a timeline chart.

5. The system of claim 1, wherein the one or more hardware processors are configured such that semantically analyzing the video content includes automatically generating the query or survey and a recommended timestamp for effectuating presentation of the automatically generated query or survey.

6. A method for effectuating presentation of video content based on complexity of video content segments therein with a system including one or more hardware processors configured by machine-readable instructions, the method comprising:
analyzing the video content using semantic ontology to identify semantic concepts in the video content, an individual semantic concept indicated by a plurality of linked keywords corresponding to an individual topic of the video content;
segmenting the video content into one or more video content segments based on the semantic concepts;
determining a measure of complexity of the one or more video content segments based on a weightage of the identified semantic concepts, the weightage of the identified semantic concepts determined based on one or more of:
types or numbers of links associated with the identified semantic concepts,
numbers of concept nodes associated with the identified semantic concepts, the number of concept nodes indicating a number of other semantic concepts relating a given semantic concept,
an education level corresponding to the user,
evaluation results of the user responding to a query or survey relating to the content, or
a clinical medical condition of the user; and
effectuating presentation of the one or more video content segments to the user based on the measure of complexity.

7. The method of claim 6, wherein determining the measure of complexity of the video content comprises adding the number of the concept nodes to a sum of a complexity measure of each of the identified semantic concepts, the complexity measure of each of the identified semantic concepts determined by increasing a weightage of each of the identified semantic concepts by one.

8. The method of claim 6, wherein effectuating presentation of the one or more video content segments includes one or more of rearranging, altering, modifying, fragmenting, combining, or replacing the one or more video content segments.

9. The method of claim 6, further comprising, responsive to the determination of the measure of complexity of the identified semantic concepts and a timestamp corresponding to the identified semantic concepts, effectuating presentation of a visualization of the measure of complexity, the visualization including a statistical probability graph, a bar chart, or a timeline chart.

10. The method of claim 6, wherein semantically analyzing the video content includes automatically generating the query or survey and a recommended timestamp for effectuating presentation of the automatically generated query or survey.

11. A system for effectuating presentation of video content based on complexity of video content segments therein, the system comprising:
means for analyzing the video content to identify semantic concepts in the video content, an individual semantic concept indicated by a plurality of linked keywords corresponding to an individual topic of the video content;
means for segmenting the video content into one or more video content segments based on the semantic concepts;
means for determining a measure of complexity of the one or more video content segments based on a weightage of the identified semantic concepts, the weightage of the identified semantic concepts determined based on one or more of:
types or numbers of links associated with the identified semantic concepts,
numbers of concept nodes associated with the identified semantic concepts, the number of concept nodes indicating a number of other semantic concepts relating a given semantic concept,
an education level corresponding to the user,
evaluation results of the user responding to a query or survey relating to the content, or
a clinical medical condition of the user; and
means for effectuating presentation of the one or more video content segments to the user based on the measure of complexity.

12. The system of claim 11, wherein the means for determining the measure of complexity of the video content comprises means for adding the number of the concept nodes to a sum of a complexity measure of each of the identified semantic concepts, the complexity measure of each of the identified semantic concepts determined by increasing a weightage of each of the identified semantic concepts by one.

13. The system of claim 11, wherein the means for effectuating presentation of the one or more video content segments includes one or more of means for rearranging, altering, modifying, fragmenting, combining, or replacing the one or more video content segments.

14. The system of claim 11, further comprising, responsive to the determination of the measure of complexity of the identified semantic concepts and a timestamp corresponding to the identified semantic concepts, means for effectuating presentation of a visualization of the measure of complexity, the visualization including a statistical probability graph, a bar chart, or a timeline chart.

15. The system of claim 11, wherein the means for semantically analyzing the video content includes means for automatically generating the query or survey and a recommended timestamp for effectuating presentation of the automatically generated query or survey.
